(19) United States
(12) Patent Application Publication
(10) Pub. No.: US 2010/0100120 A1
(43) Pub. Date: Apr. 22, 2010

(54) DEVICES AND METHODS FOR CONTROLLING BLEEDING

(75) Inventors: Jessica Perkins, Southern Pines, NC (US); Corey Russ, Fayetteville, NC (US); Christopher Murphy, Vass, NC (US)

Correspondence Address: KNOBBE MARTENS OLSON & BEAR LLP, 2040 MAIN STREET, FOURTEENTH FLOOR, IRVINE, CA, 92614, US

(73) Assignee: COMBAT MEDICAL SYSTEMS, LLC, Fayetteville, NC (US)

(21) Appl. No.: 12/604,304

(22) Filed: Oct. 22, 2009

Related U.S. Application Data
(60) Provisional application No. 61/107,362, filed on Oct. 22, 2008.
(60) Provisional application No. 61/221,005, filed on Jun. 26, 2009.

(51) Int. Cl. A61B 17/12

(52) U.S. Cl. 606/201

(57) ABSTRACT

A compression or tourniquet device can include a frame which has a first arm and a support arm and a pad mounted on the first arm. The position of the first arm can be laterally and vertically adjustable on the frame relative to the brace arm. The device can be secured to a patient and the pad can be adjusted to provide pressure on an injury to stop the bleeding.

### RELATED APPLICATIONS

The present application claims priority under 35 U.S.C. §119(e) to U.S. Provisional Application No. 61/107,362 filed on Oct. 22, 2008 and U.S. Provisional Application No. 61/221,005 filed on Jun. 26, 2009, the disclosures of which are incorporated by reference herein in their entirety.

### FIELD OF THE DISCLOSURE

This disclosure relates to medical devices, and in particular to devices for controlling bleeding.

### BACKGROUND OF THE DISCLOSURE

The rapid control of bleeding at wound sites can be important in wound management, especially for the management of trauma, e.g., as may result from military exercises, automobile accidents, industrial accidents, etc. Conventional approaches such as manual pressure, cauterization, or sutures may be time-consuming and are not always effective in controlling bleeding. Trauma care has received great attention recently as military personnel on a daily basis face combat situations that result in wounds accompanied by significant blood loss. In many cases, the individual may survive the initial injury only to die of blood loss. Even where manual pressure is effective to control bleeding, the application of manual pressure requires the constant attention of medical personnel who are thereby unavailable to help others.

### SUMMARY OF THE DISCLOSURE

The present disclosure relates generally to hemorrhage control caused by trauma or other conditions. In some embodiments, a bleeding control device, such as a compression or tourniquet device, can include a frame which has a first arm and a brace arm and a pressure pad mounted on the first arm. The position of the first arm can be laterally and vertically adjustable on the frame relative to the brace arm.

### BRIEF DESCRIPTION OF THE DRAWINGS

FIG. 1 is an exploded schematic view of a bleeding control device according to an embodiment of the invention.

FIG. 2 is a perspective view of an assembled bleeding control device according to another embodiment of the invention.

FIG. 3 is an exploded schematic view of the bleeding control device of FIG. 2.

FIG. 4 is a perspective view of the bleeding control device of FIG. 2 in a transport configuration.

FIGS. 5A-C are various views of the extender of FIG. 3.

FIGS. 6A-D are various views of the sleeve adapter of FIG. 3.

FIGS. 7A-B are various views of the extender and sleeve adapter assembly of FIGS. 5A-C and FIGS. 6A-D.

FIGS. 8A-B are various views of the riser sleeve of FIG. 3.

FIGS. 9A-B are side views of the riser of FIG. 3.

FIG. 10 is a top view of an embodiment of a projection on a riser sleeve.

FIG. 11 is a close-up side view of the bleeding control device of FIG. 2 illustrating the spindle, first arm, sleeve member, sleeve screw and riser.

FIGS. 12A-C are various views of the first arm of FIG. 3.

FIG. 13 is a perspective top view of a ring coupled to the first arm of FIGS. 12A-C.

FIGS. 14A-B are various views of the spindle of FIG. 3.

FIGS. 15A-B are various views of the handle of FIG. 3.

FIGS. 16A-C are various views of the head of the spindle of FIG. 3.

FIGS. 17A-B are various views of the spindle assembly of FIG. 3 illustrating the spindle, handle and head of the spindle.

FIG. 18 is a perspective view of the pressure pad of FIG. 3.

FIGS. 19A-C are various views of the pressure pad of FIG. 3.

FIGS. 20A-C are various views of a pressure pad according to another embodiment of the invention.

FIGS. 21A-C are various views of a pressure pad according to another embodiment of the invention.

FIG. 22 is a perspective view of the brace arm of FIG. 2.

FIGS. 23A-D are various views of the brace arm of FIG. 3.

FIGS. 24A-B are various views of the brace bushing of FIG. 3.

FIG. 25 is a perspective view of an embodiment of a bleeding control device.

FIG. 26A is a perspective view of a component of a bleeding control device.

FIG. 26B is a side view of the component of FIG. 26A.

### DETAILED DESCRIPTION OF CERTAIN EMBODIMENTS

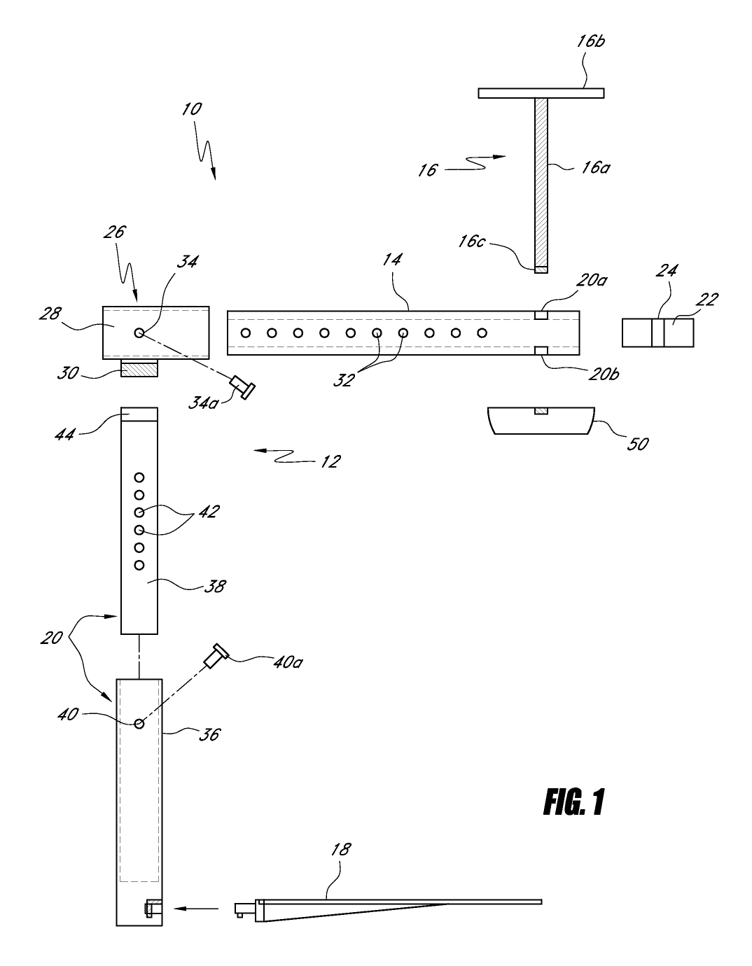

A bleeding control device, such as a compression or tourniquet device, according to an embodiment, as indicated generally by the numeral 10 in FIG. 1, can include a frame 12 that is generally c-shaped (e.g., having two generally parallel sides that are generally perpendicular to another side). The frame 12 can include a first arm 14 which carries a spindle 16, and a brace arm 18. The first arm 14 and the brace arm 18 can be joined by a riser 20.

In the illustrated embodiment, the first arm 14 is a hollow member which can be equipped with spindle passage apertures 20a, 20b and which can receive a connector (e.g., a nut) insert 22 therein. The spindle passage apertures 20a, 20b are configured to permit the spindle 16 to pass through the first arm 14. The connector insert 22 can comprise a threaded passage 24. The spindle 16 can have a shaft 16a with an external screw thread, and the threaded passage 24 can be configured to engage the external screw thread of the spindle 16. The spindle 16 can have a handle 16b to facilitate turning the spindle to advance or retract the spindle through the connector insert 22.

The frame 12 can include a sleeve adapter 26 which comprises a sleeve member 28 and a mounting stud 30. The sleeve member 28 can be configured to receive the first arm 14 therethrough in an adjustable (e.g., a telescoping-type) arrangement. The first arm 14 can include a plurality of positioning apertures 32, and the sleeve member 18 can have an anchoring aperture 34. In some embodiments, the relative position of the first arm 14 in the sleeve member 28 can be fixed at any one of a plurality of positions by aligning the anchoring aperture 34 with a selected positioning aperture 32 and inserting a securing pin 34a through the mutually aligned anchoring aperture and positioning aperture. In this way, the position of the first arm 14 is generally laterally (e.g., generally horizontally) adjustable on the riser 20 relative to the brace arm 18 and/or the distance from the wound contract portion 50 to the riser 20 is configured to be selectively increased or decreased.

The riser 20 can include a riser sleeve 36 and an extender 38, and the riser sleeve can be configured to receive the extender therein in an adjustable (e.g., telescoping-type) arrangement. The riser sleeve 36 can have a riser sleeve anchor aperture 40 and the extender 38 can include a plurality of positioning apertures 42. In some embodiments, the relative position of the extender 38 in the riser sleeve 36 can be fixed at any one of a plurality of positions by aligning the riser sleeve anchor aperture 40 with a selected positioning aperture 42 and inserting a securing pin 40a through the mutually aligned riser sleeve anchor aperture and positioning aperture. In this way, the length of the riser 20 is adjustable, making the position of the first arm 14 on the frame 12 generally vertically adjustable relative to the brace arm 18. A first riser end region 44 can engage the mounting stud 30 so that the riser 20 extends from the sleeve adapter 26 at an angle (e.g., at a right angle) relative to the first arm 14. The brace arm 18 can be secured to the riser 20 at a second end region 48 of the riser, and can extend from the riser at an angle so that the brace arm is generally parallel to the first arm 14. Preferably, the brace arm 18 is configured to have a flat surface that faces the first arm 14, so that the brace arm can be more comfortably situated against a patient's body.

In some embodiments, to assemble the bleeding control device 10, the nut insert 22 is inserted into the first arm 14 with the threaded passage 24 aligned with the spindle passage apertures 20a, 20b, and the shaft 16a of the spindle 16 is passed through the spindle passage apertures 20a, 20b and the threaded passage 24. Once the head 16c of the shaft 16a emerges from the spindle passage aperture 20b, a wound contact portion (e.g., a pressure pad) 50 can be mounted onto the head of the shaft in a way that leaves the pressure pad freely rotatable on the shaft. In some embodiments, the wound contact portion 50 can directly contact the bleeding wound or the wound contact portion can contact other materials (such as bandages or clothing that in turn contact the bleeding wound). By moving (e.g., rotating) the shaft 16a, the pressure pad 50 can be advanced or retracted relative to the brace arm 18.

In some embodiments, the first arm 14 can be positioned in the sleeve member 28 with the anchoring aperture 32 aligned with a selected positioning aperture 34, and the securing pin 34a can be inserted through the aligned apertures to secure the position of the first arm in the sleeve adapter 26. The brace arm 18 can be mounted on the riser sleeve 36, and the length of the riser 20 can be fixed by aligning the riser sleeve anchor aperture 40 with a desired positioning apertures 42, and inserting a securing pin 40a through the aligned apertures. The initial distance (e.g., the distance when compression or pressure is initiated) between the wound contact portion 50 and the brace arm 18 can be configured to be selectively increased or decreased. The riser 20 can be mounted on the sleeve adapter 26 by securing the first riser end region 44 onto the mounting stud 30.

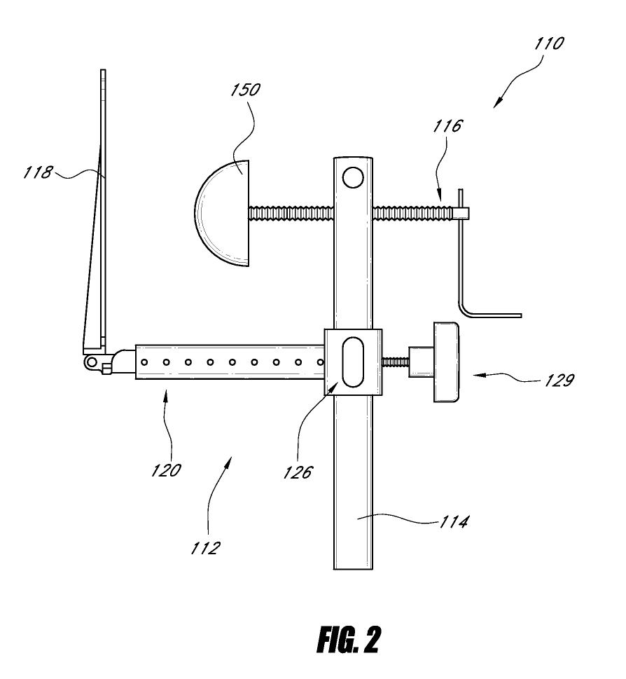

FIG. 2 shows another embodiment of a compression or tourniquet device. In this and other illustrated embodiments, the components are labeled with corresponding reference numbers as the embodiments described above, except added to one hundred. For example, the embodiment described above is a compression or tourniquet device labeled as 10. FIG. 2, illustrating another embodiment of a compression or tourniquet device, is labeled as 110.

As illustrated in the embodiment of FIG. 2, the compression or tourniquet device 110 can comprise a frame 112 that is generally C-shaped in the assembled configuration. The frame 112 can include a first arm 114 which carries a spindle 116 having a pressure pad 150, and a brace arm 118. The first arm 114 and the brace arm 118 can be joined by a riser 120. Additional figures and disclosure below provide further information about this embodiment.

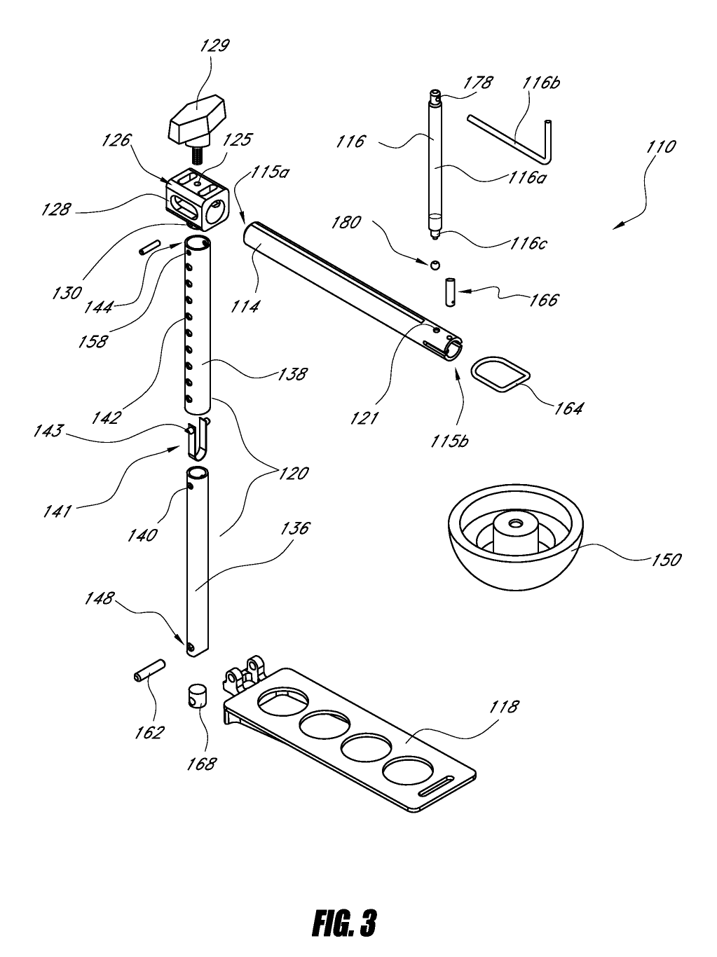

FIG. 3 illustrates an exploded perspective view of the compression or tourniquet device 110 according to an embodiment of the invention. The compression or tourniquet device 110 can include a riser 120, comprising a riser sleeve 136 and an extender 138 that are coupled together by a projection 143. The extender 138 can be configured to receive the riser sleeve 136 therein in an adjustable (e.g., a telescoping-type) arrangement. In the illustrated embodiment of FIG. 3, the riser sleeve 136 and extender 138 are elongate tubes. A sleeve adapter 126 can be attached to the first end region 144 of the riser 120 and a brace arm 118 can be attached to the second end region 148 of the riser 120. The brace arm 118 can be a generally flat support surface that is disposed generally perpendicular to the riser 120. As illustrated in FIG. 3, a first arm 114 can be an elongate tube that extends generally perpendicular to the riser 120 and can be slideably coupled to the sleeve adapter 126. In some embodiments, the first arm 114 can be generally parallel to the brace arm 118. At or near an end region of the first arm 114 can be a spindle 116 that is threadedly engaged to the first arm 114. Connected to the spindle 116, as at an end region of the spindle 116, can be a pressure pad 150 whose distance from the brace arm 118 can be selectively adjusted, such as by screwing, rotating, and/or sliding the spindle 116.

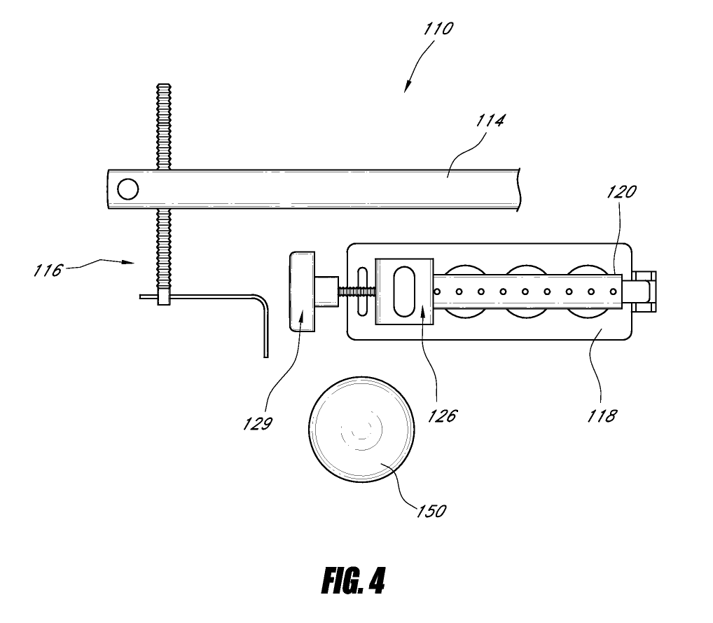

With reference to FIG. 4, in some embodiments, the compression or tourniquet device 110 can be arranged in a folded configuration for easier transport (for example, to facilitate transport by military personnel in circumstances where there are many size and weight constraints). The first arm 114 can be detached from the sleeve adapter 126 and the pressure pad 150 can be detached from the spindle 116. The brace arm 118 can pivot at the second end region 148 of the riser 120 and swing until the brace arm 118 is substantially parallel with the riser 120. In the embodiment illustrated in FIG. 4, the compression or tourniquet device 110 is separated into multiple (e.g., three) pieces in the transport configuration. In other embodiments, the compression or tourniquet device 110 can remain a single piece in the transport configuration. For example, the sleeve adapter 126 can be pivotally coupled to the riser, such that the first arm 114 can fold against the riser 120 and the pressure pad 150 can be configured to remain on the spindle 116 in the transport configuration. In other embodiments, the compression or tourniquet device 110 can comprise any number of pieces in the transport configuration.

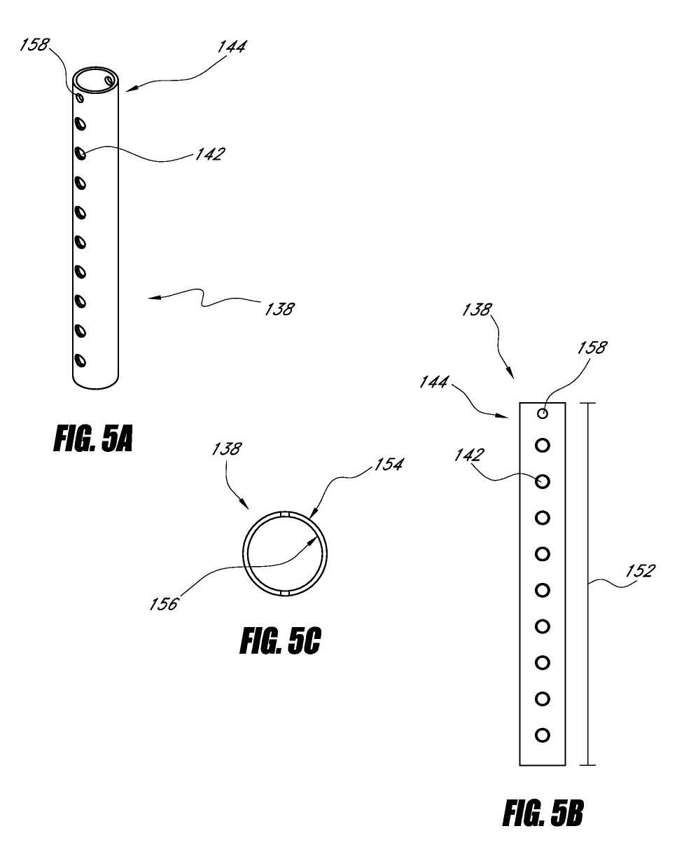

As illustrated in FIG. 5, the extender 138 can be an elongate tube having a length 152, an outer diameter 154 and an inner diameter 156. Preferably, the length 152 of the extender 138 has a range from greater than or equal to about 4 inches and/or less than or equal to about 8 inches. In the illustrated embodiment, the length 152 of the extender 138 is 6 inches. In other embodiments, the length 152 can be any desired size. In a preferred embodiment, the outer diameter 154 is preferably approximately 0.75 inches and the inner diameter 156 is approximately 0.634 inches. In some embodiments, the outer diameter 154 can range from greater than or equal to about 0.50 inches and/or less than or equal to about 1.0 inches. In some embodiments, the inner diameter 156 can range from greater than or equal to about 0.25 and/or less than or equal to about 0.90 inches. In other embodiments, the outer diameter 154 and inner diameter 156 can be any other size.

The extender 138 can have one or more positioning apertures 142 that can be used in adjusting the length of the riser 120. The positioning apertures 142 are disposed at discrete intervals along the length of the extender 138. In some embodiments, positioning apertures 142 are disposed on opposite sides of the extender 138. In some embodiments, the positioning apertures 142 are disposed on one side of the extender 138. As described in more detail below, in some embodiments, the positioning apertures can accept projections 143 for fixing the length of the riser 120. In some embodiments of the extender 138, an adapter mounting aperture 158 can be positioned toward the first end region 144. The adapter mounting aperture 158 can be threaded to accept a screw or can be a thru hole to accept a pin. In some embodiments, the length of the riser 120 is configured to be adjustable to assist in positioning the bleeding control device close to a human body in a manner that generally surrounds at least a portion of a region (such as the torso or an appendage) where effusive bleeding is occurring. The sizes and range of adjustment in the riser 20, 120 and/or the first arm 14, 114 can be configured to permit the pressure pad 150 to be positioned and/or oriented across a human body to access a bleeding site. Once the bleeding control device is so positioned, the location of the pressure pad 150 with respect to the first arm 114 can be adjusted so as to exert a focused pressure on the bleeding wound. In some embodiments, the pressure pad 150 can be fixedly mounted on the first arm 114 and the adjustment of the length of the riser 120 can provide the pressure on the wound. In some embodiments, both the adjustment of the length of the riser 120 and the relative position between the first arm 114 and the pressure pad 150 can provide the pressure on the wound.

### Riser Sleeve

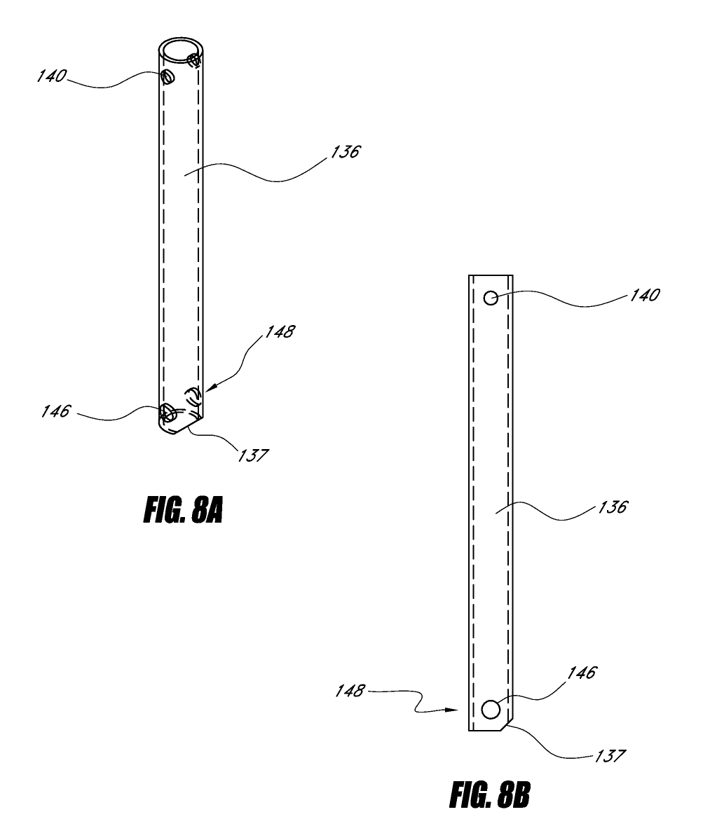

With continued reference to FIG. 3, the riser 120 can comprise a riser sleeve 136 that is slideably coupled with the extender 138 in an adjustable (e.g., a telescoping-type) arrangement. In the illustrated embodiment of FIGS. 8A-B, the riser sleeve 136 is an elongate tube with a brace connection aperture 146 toward the second end region 148 and a riser sleeve anchor aperture 140 toward the other end. In a preferred embodiment, the riser sleeve 136 is approximately 6.5 inches in length. In other embodiments, the riser sleeve 136 can be any desired length. At the second end region 148, the riser sleeve 136 can have a chamfer 137 on a side to facilitate the rotation of the brace arm 118, as discussed later.

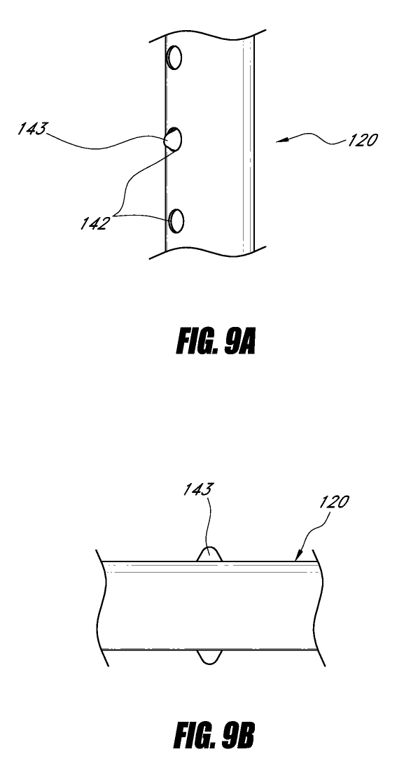

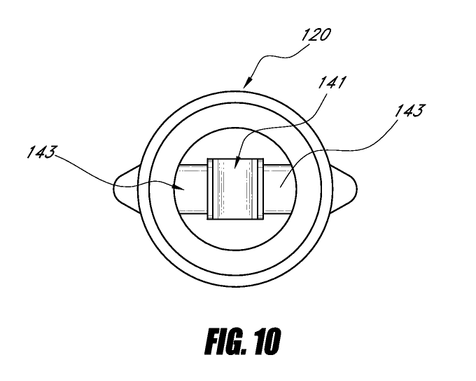

As illustrated in the embodiment of FIG. 3, the riser sleeve 136 can slide inside of the extender 138 and the riser sleeve 136 and extender 138 can be fixed by means of a holding member 141, which is positioned inside of the riser sleeve 136. In some embodiments, the holding member 141 can comprise a spring that biases projections 143 in an outward direction extending through the riser sleeve anchor aperture 140 and projecting outwardly through one of the positioning apertures 142 on the extender 138, as illustrated, for example, in FIGS. 9A-B. The relative position of the extender 138 in the riser sleeve 136 can be fixed at any one of a plurality of positions by aligning the holding member 141 with a selected positioning aperture 142 on the extender 138 so that the projections 143 can extend through the selected positioning aperture 142 and fix the relative positions of the riser sleeve 136 and extender 138. To adjust the length of the riser 120, the projections 143 can be squeezed together, as illustrated in FIG. 10, to disengage the holding member 141 from the selected positioning aperture 142, and then moved to a new positioning aperture 142. In some embodiments, the effective length of the riser 120 can be adjusted by including a pivoting portion (instead of, or in addition to, other length adjusting mechanisms such as a telescoping feature as illustrated) to permit discrete sections of the riser 120 to be oriented at an angle such that the respective end regions of these sections of the riser 120 are brought closer together. Thus, the length of the riser 120 can be adjustable, making the position of the first arm 114 generally vertically adjustable relative to the brace arm 118.

### Sleeve Adapter

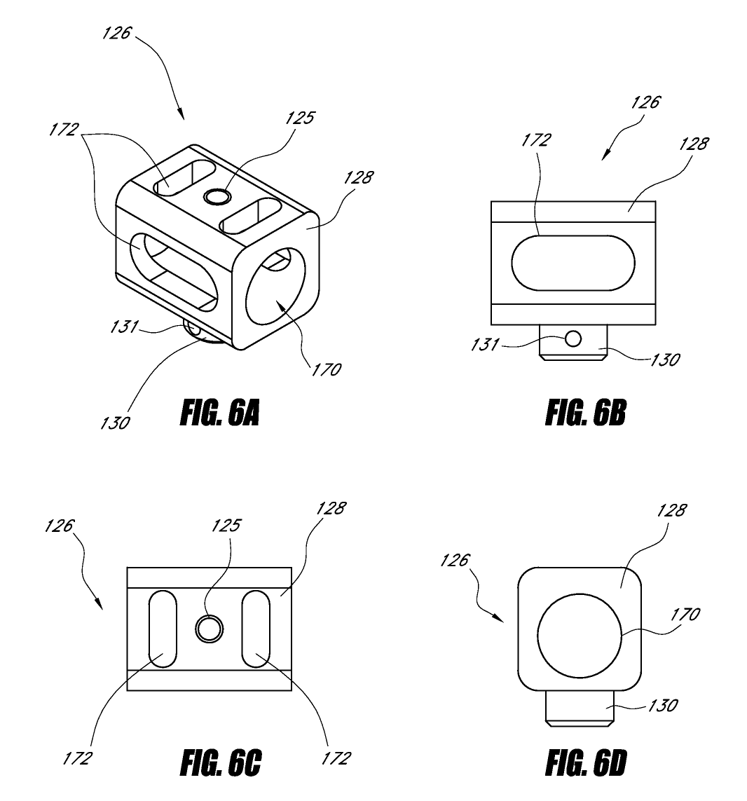

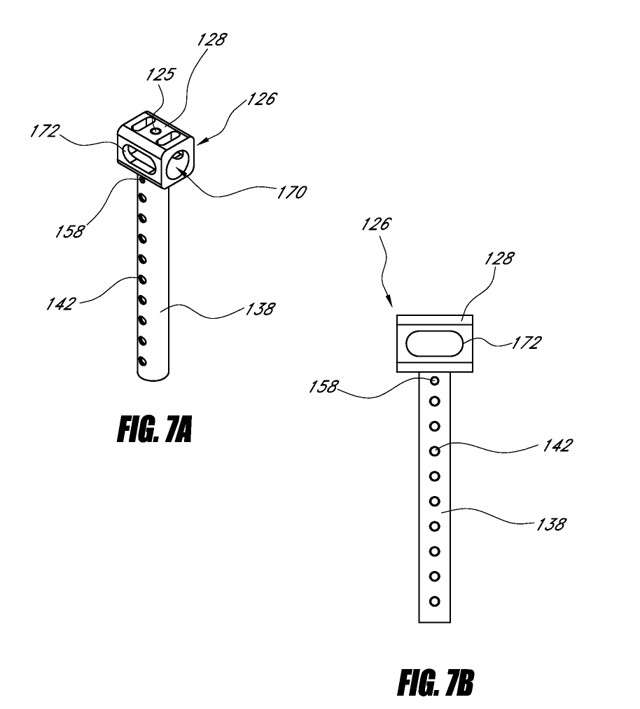

A first end region 144 of the riser 120 can be coupled to a sleeve adapter 126. The sleeve adapter 126 can comprise a sleeve member 128 and a mounting stud 130. The mounting stud 130 can be configured to attach with the extender 138. In the embodiment illustrated in FIGS. 6A-D, the mounting stud 130 is a circular protrusion that fits into the inner diameter 156 of the extender 138 at the first end region 144. In some embodiments, the mounting stud 130 can be secured to the inner diameter 156 through an interference fit. In other embodiments, the connection between the mounting stud 130 and the first end region 144 can be secured by any other means, such as fasteners, welding or adhesives. In some embodiments, the mounting stud 130 can comprise a pin anchoring aperture 131 for accepting a first pin 160, as illustrated in FIGS. 7A-B. In these embodiments, the pin anchoring aperture 131 can be aligned with the adapter mounting aperture 158 on the extender and a first pin 160 can be inserted through the aligned apertures. The first pin 160 can be secured in the apertures 131, 158 through any means known in the art, such as for example welds or adhesives. In a preferred embodiment, the first pin 160 is press or interference fitted into the apertures 131, 158. In other embodiments, the first pin 160 can be a screw that fastens to threads on the pin anchoring aperture 131 and/or adapter mounting aperture 158.

With continued reference to FIGS. 6A-D, the sleeve member 128 can be configured to receive the first arm 114 therethrough, wherein the sleeve member 128 can be adjustably positioned along the length of the first arm 114. The sleeve member 128 can comprise a clearance bore 170 that extends laterally through the length of the sleeve member 128 through which the first arm 114 can slide. In some embodiments, the bore can be cylindrical in shape. In other embodiments, the bore 170 can have a shape complimentary to the outer shape of the first arm 114, as illustrated in FIG. 6D. In some embodiments, the bore 170 can be any shape that provides a clearance fit for the first arm 114. In some embodiments, the sleeve member 128 can comprise side holes 172 on the walls of the sleeve adapter. The side holes 172 can advantageously reduce the weight of the component, which can be useful when the compression or tourniquet device is carried long distances in the field.

In some embodiments, the sleeve member 128 can be integral to the extender 138 wherein the sleeve member 128 and the extender 138 are made of a single piece of material. In some embodiments, the extender 138 can extend from the sleeve member 128 at an angle (e.g., generally at a right angle) relative to the first arm 114. In other embodiments, the extender 138 can extend from the sleeve member 128 at an acute, obtuse, or oblong angle relative to the first arm 114.

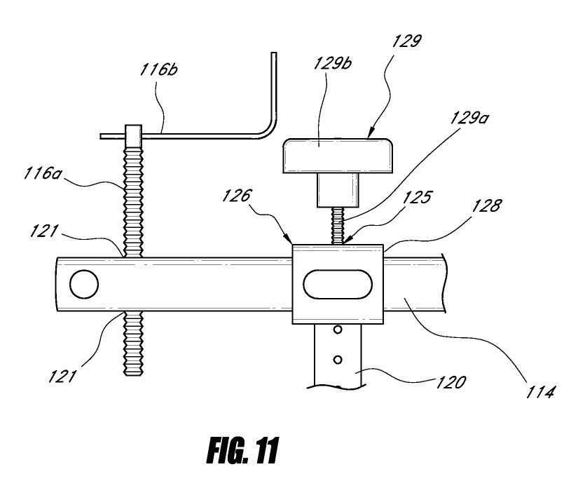

As illustrated in FIG. 11, the first arm 114 can be slideably coupled to the sleeve adapter 126. The sleeve member 128 can include a means for fixing the first arm 114 to the sleeve adapter 126. In the embodiment illustrated in FIG. 11, a sleeve screw 129 can fix the first arm 114 to the sleeve adapter 126. For example, the sleeve member 128 can include a sleeve screw aperture 125 having threads that are complimentary to the threads on the sleeve screw 129. The relative position of the first arm 114 in the sleeve member 128 can be fixed at any position by tightening the sleeve screw 129 in the sleeve screw aperture 125 until the end region of the sleeve screw 129 presses against the first arm, fixing the position of the first arm 114 within the sleeve member 128.

A handle 129b can be attached to a first end region of the threaded shaft 129a. The handle 129b can be T-shaped, as shown in FIG. 11. In some embodiments, the handle can be a round knob. In other embodiments, the handle can be an L-shaped rod. The second end region of the threaded shaft 129a can be configured to engage the first arm 114. For example, the second end region can be pointed, textured, or jagged so that it can dig into the first arm 114 when tightened. In some embodiments, the second end region can have other configurations that can facilitate the second end region to securely engage to the first arm 114.

In some embodiments, the sleeve screw 129 can be coupled to the top of the sleeve member 128, as illustrated in FIG. 11. In other embodiments, the sleeve screw 129 can be coupled to a side of the sleeve member 128. In still other embodiments, the sleeve screw 129 can be coupled to a corner of the sleeve member 128.

### First Arm

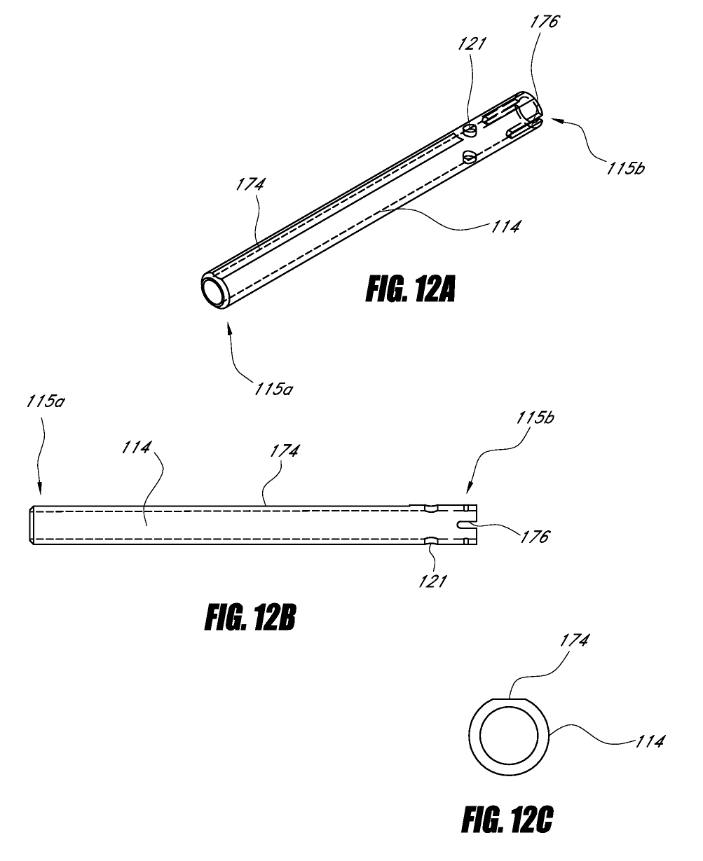

As illustrated in the embodiment of FIG. 12, the first arm 114 can be an elongate tube with a first end region 115a and a second end region 115b. In a preferred embodiment, the first arm 114 is approximately 10 inches in length. In some embodiments, the first arm 114 can range from greater than or equal to about 5 inches and/or less than or equal to about 15 inches. In general, the dimensions provided herein are intended to allow the bleeding control devices to fit around and apply compression or pressure to a portion of a human body.

In the illustrated embodiment, the first arm 114 comprises a flat portion 174 that extends along the length of the first arm 114 from the first end region 115a to a distance of approximately 8.25 inches from the first end region 115a. In some embodiments, the length of the flat portion 174 can extend generally the entire length of the first arm 114. In other embodiment, the length of the flat portion 174 can range from greater than or equal to about 1 inch and/or less than or equal to about 10 inches. In the illustrated embodiment, the depth of the flat portion 174 is approximately 0.021 inches. However, the depth of the flat portion 174 can be any depth sufficient enough to provide an engagement surface for the sleeve screw without compromising the structural integrity of the first arm 114. In some embodiments, the first arm 14, 114 can comprise one or more angled, pivoting, or articulating portions that permit the pressure pad 50, 150 to be oriented at a non-perpendicular angle relative to the first arm 14, 114 to permit pressure to be applied to a wound in a position on the human body that may be difficult to reach in a generally perpendicular orientation between the components as illustrated.

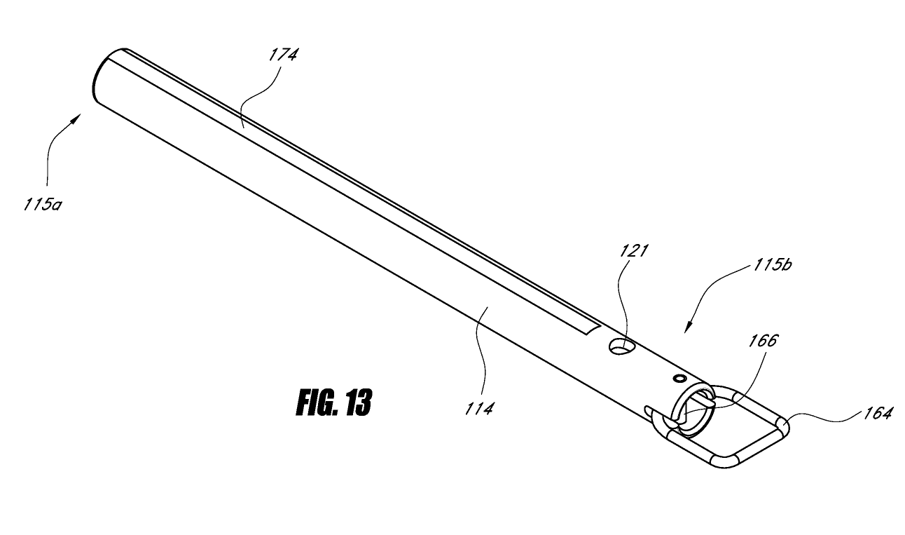

In some embodiments, the first arm 114 can comprise openings or cutouts 176 at the second end region 115b for attaching a ring 164. The ring 164 can be inserted into the cutouts 176 and secured with a third pin 166, as illustrated in FIGS. 3 and 13. The ring 164 can be used to secure or hang the first arm 114 during transport. The ring 164 can advantageously be utilized as a handle when slideably adjusting the first arm 114 relative to the sleeve adapter 126. In some embodiments, the ring 164 can be generally D-shaped (e.g., having a generally straight side and a generally curved side) and formed from an elongate member having a circular cross-section. In some embodiments, the ring 164 can have other shapes, such as a round or oval shape. In some embodiments, the ring's cross-sectional shape may not be circular, such as for example a square cross-section. In some embodiments, the ring 164 can be a closed loop or the ring can be an open loop. For example, the ring can be a carabineer, which comprises a latch that can open in one direction for securing to other objects, but does not open in the opposite direction

In some embodiments, the sleeve member 128 can be coupled with the first arm 114 through a plurality of positioning apertures, as described above. The relative position of the first arm 114 in the sleeve member 128 can be fixed at any one of a plurality of positions by aligning the sleeve screw aperture 125 with a selected positioning aperture disposed on the first arm 114 and inserting a securing pin through the mutually aligned sleeve screw aperture 125 and positioning aperture. In some embodiments, a sleeve screw 129 can be inserted into the mutually aligned sleeve screw aperture 125 and positioning aperture.

### Spindle

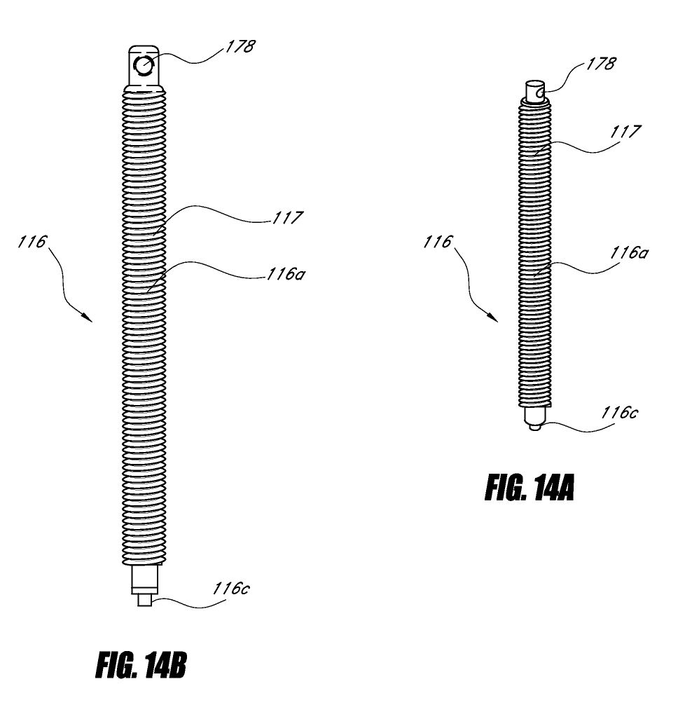

In some embodiments, the first arm 114 can be equipped with spindle passage apertures 121 toward the second end region 115b of the first arm 114. As illustrated in FIGS. 14A-B, a spindle 116 can have a shaft 116a with an external thread 117. The spindle passage apertures 121 can have threads that are configured to engage the external thread 117 on the shaft 116a. The spindle 116 can be adjusted by rotating the spindle 116.

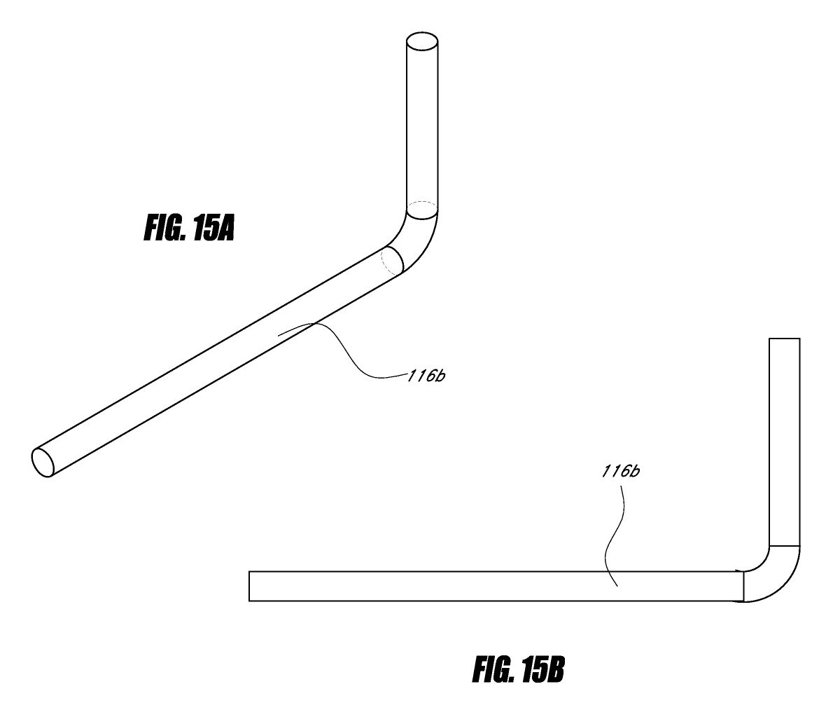

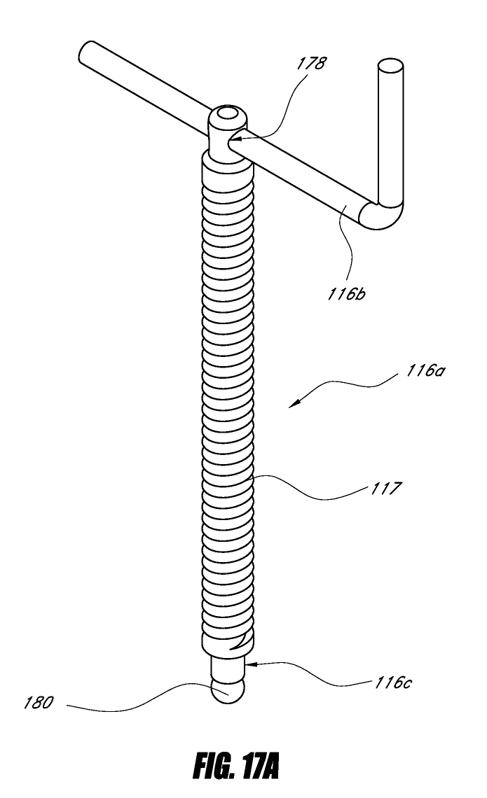

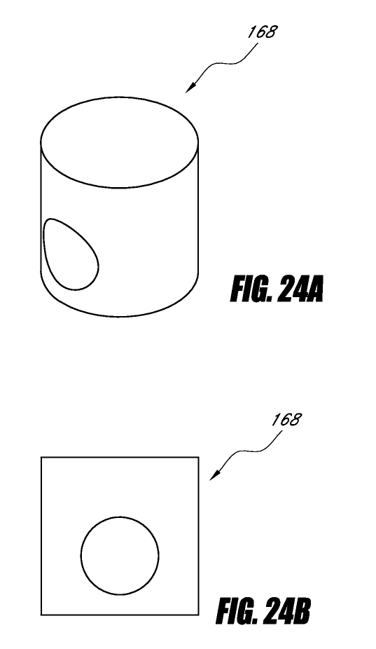

The spindle 116 can have a handle 116b to facilitate turning or otherwise moving the spindle to advance or retract the spindle 116 through the first arm 114. In some embodiments, an end region of the shaft 116a can comprise a handle mounting hole 178 where the handle 116b can mount. In some embodiments, the handle 116b can be a straight member. In other embodiments, as illustrated in FIGS. 15A-B the handle 116b can be L-shaped (e.g., having a first side generally perpendicular to a second side), which can enable a user to rotate the spindle 116 quickly by grabbing one leg of the L-shaped handle to rotate the spindle 116. In some embodiments, the handle can have a grip at an end region of the handle, such as for example the ball illustrated in FIG. 25. In the illustrated embodiment of FIGS. 17A-B, the L-shaped handle 116b is inserted in the handle mounting hole 178 and the handle 116b is crimped on one side of the shaft 116a to prevent the handle 116b from uncoupling from the shaft 116a. In other embodiments, the handle 116b can be secured to the shaft 116a through other means, such as for example fasteners, welding, or interference fits. In some embodiments, the handle mounting hole 178 and the handle 116b can have complimentary shapes that substantially prevent the handle 116b from freely spinning within the handle mounting hole 178. For example, the handle 116b can have a square cross-sectional area and the handle mounting hole 178 can be square shaped so that the handle 116b cannot freely rotate within the handle mounting hole 178.

In some embodiments, the spindle passage apertures 121 can be clearance holes that are configured to permit the spindle 116 to pass through the first arm 114. A nut insert, as described in another embodiment above, comprising a threaded passage can be provided that is inserted into a hollow portion of the first arm 114. The threaded passage can be configured to engage the external screw thread of the spindle 116.

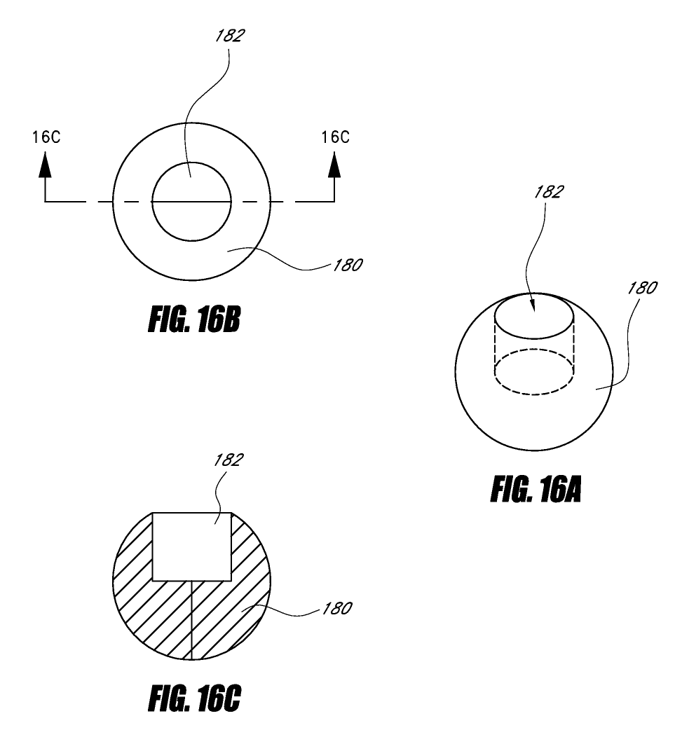

The head of the spindle 116c can have a spindle ball 180, as illustrated in FIGS. 17A-B, for coupling with a pressure pad 150. In the embodiment illustrated in FIGS. 16A-C, the spindle ball 180 is a sphere or generally spherical with a cylindrical cavity 182 for accepting the head of the spindle 116c. The head of the spindle 116c and the spindle ball 180 can be coupled together through an interference fit or through other means of attachment, such as welding, threading, or adhesives. In a preferred embodiment, the spindle ball 180 is made of a metal, such as for example stainless steel. In other embodiments, the spindle ball 180 can be made of any rigid material such as composites or hard plastics.

### Pressure Pad

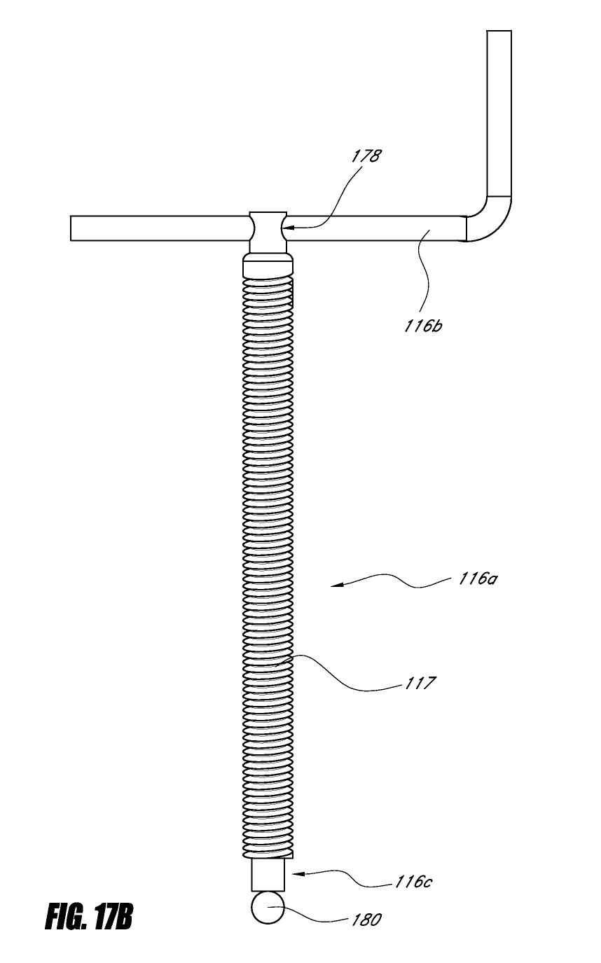

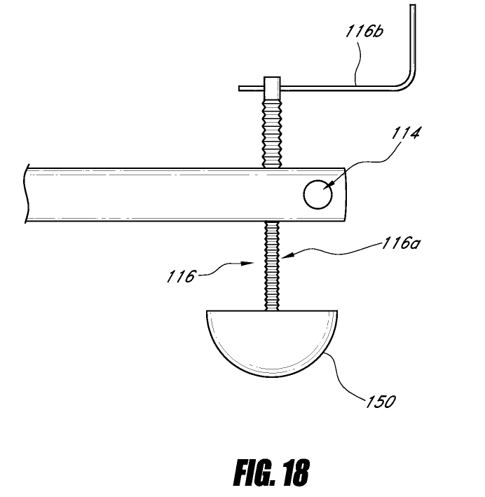

With reference to FIG. 18, a pressure pad 150 can be attached to the shaft 116a of the spindle 116. The pressure pad 150 can distribute the pressure from the compression or tourniquet device 110 to the patient's body. As illustrated in the embodiment of FIGS. 19A-C, the pressure pad 150 can be a semi-spherical shape with the rounded side facing toward the brace arm 118 and a flat top side coupled to the shaft 116a. In some embodiments, the top has a shape other than substantially flat. Preferably, the pressure pad 150 had a diameter of approximately 3 inches. In other embodiments, the pressure pad 150 can have a diameter ranging from greater than or equal to about 1 inch and/or less than or equal to about 5 inches. In some embodiments (as illustrated), the pressure pad 150 can include a surface configured to exert pressure in the area of the bleeding wound that is generally curved or rounded (e.g., with a convex curve) and/or has no corners, edges, or abrupt changes in shape on the surface of the pressure pad 150 to avoid causing discomfort or additional trauma (such as skin perforations) to the patient in the region surrounding the bleeding wound. In some embodiments, the pressure pad 150 can be made of a rigid metal, plastic or composite, such as for example steel, aluminum or Delrin. In some embodiments, the pressure pad 150 can include a soft, resilient, pliable, or other partial force-absorbing and/or blood-absorbing portion to diminish the risk of causing further injury to the patient during the compression stage and/or to retain blood exiting the wound to assist in clotting. At least a portion of the pressure pad 150 can also include antimicrobial and/or blood clotting agents. The pressure pad 150 can be made of any suitable material known in the art, such as any of a variety of rigid materials. In some embodiments, the pressure pad 50, 150 can be removable from the spindle 16, 116, as shown, so that it can be replaced after each patient use. One or more pressure pads 50, 150 can be provided separately in sterilized packaging, since this portion of the bleeding control device 10, 110 is most likely to come into close contact with the patient's blood and may increase the risk of contaminant transfer. In general, the wound contacting portion or pressure pad 150 has a substantially larger wound-facing surface area than the horizontal cross-section area of the spindle 16 on which it is mounted to provide a larger area over which to apply pressure exerted by the bleeding control device.

The pressure pad 150 can have a cavity 182 toward the center with an enlarged opening 184 at the distal end of the cavity 182 for accepting the spindle ball 180. The enlarged opening 184 can be large enough to accommodate the spindle ball 180 without constraining the movement of the pressure pad 150 about the spindle ball 180. Thus, the pressure pad 150 can be configured to adjust to the anatomical shape of the patient when in use. The sides of the rest of the cavity 182 can have an interference fit with the spindle ball 180. In the embodiment illustrated in FIGS. 19A-C, the pressure pad 150 can be attached to the spindle 116 by pressing the spindle ball 180 past the sides of the cavity 182 and into the enlarged opening 184. Once the spindle ball 180 is within the enlarged opening 184, the spindle ball 180 is prevented from falling out of the cavity by the interference from the sides of the cavity 182. In other embodiments, the head of the spindle 116c can comprise a threaded end that fastens to a complimentary threaded hole in the pressure pad 150. In some embodiments, the head of the spindle 116c and the pressure pad 150 can have a press fit attachment, welded connection, or be coupled with a screw.

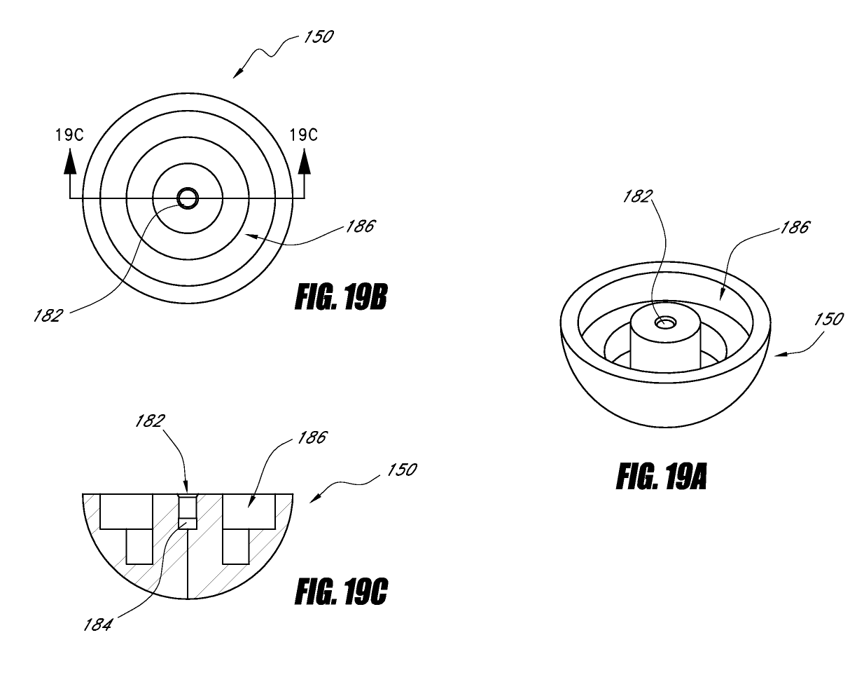

The pressure pad 150 can comprise a hollow 186, as illustrated in FIG. 19A-C. The hollow 186 can reduce the weight and cost of the pressure pad 150 for easier transport. In some embodiments, such as illustrated in FIGS. 20A-C, the pressure pad 150′ can be solid without hollows. In these embodiments, the pressure pad 150′ is preferably made of a lightweight yet rigid material, such as Delrin or aluminum.

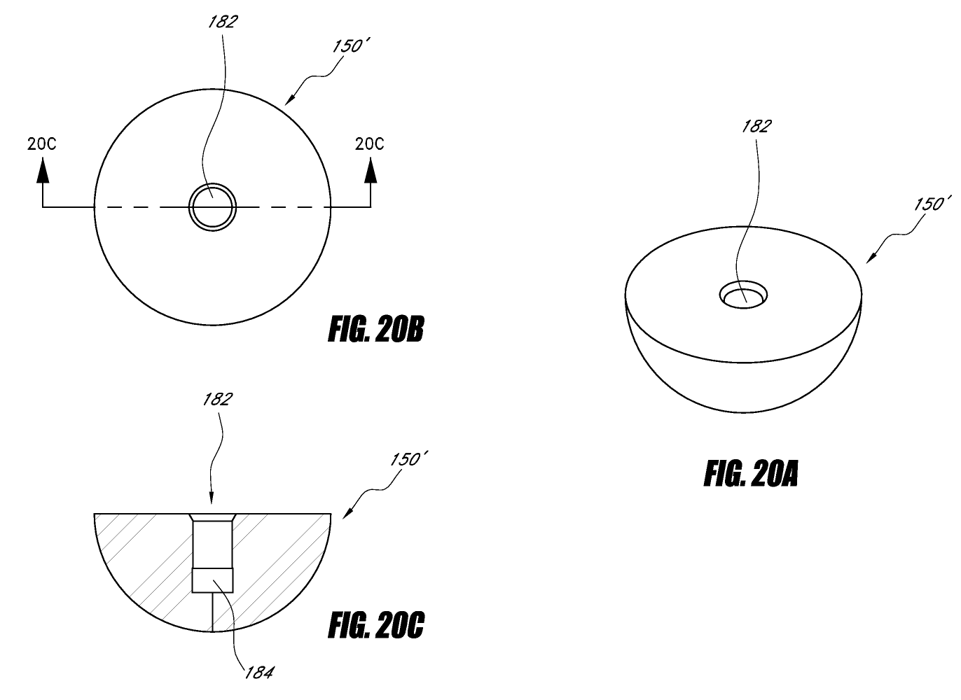

In some embodiments, the pressure pad 150″ can have shapes other than semi-spherical. For example, FIGS. 21A-C illustrate an embodiment of a rectangular pressure pad 150″. In some embodiments, the pressure pad 150″ can have a generally flat bottom. In other embodiments, the pressure pad can have a bottom that is slightly curved, or any other shape for providing sufficient pressure to an injury. In the illustrated embodiment, the pressure pad 150″ has a length of 3.5 inches and a width of 2 inches. In some embodiments, the pressure pad 150″ can have a length of 3 inches and a width of 1 inch. In other embodiments, the pressure pad 150″ can have a length of 2.5 inches and a width of 1 inch. Rectangular pressure pad 150″ having other dimensions are also contemplated. Preferably, the side of the pressure pad 150″ that contacts the patient has curved edges 188, as illustrated in FIGS. 21A and C.

### Brace Arm

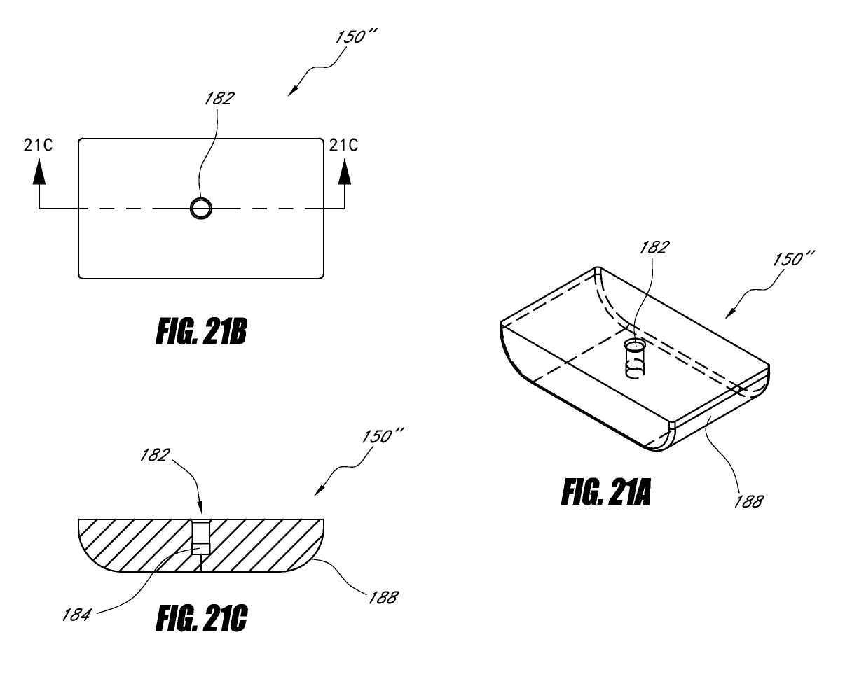

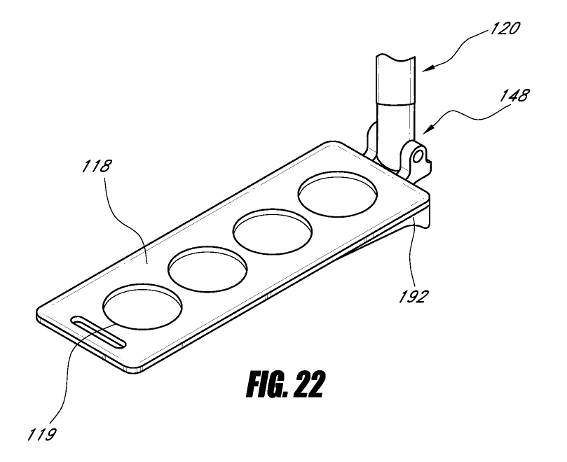

With reference to FIG. 22, the brace arm 118 can be secured to the riser 120 at a second end region 148 of the riser, and can extend from the riser 120 at an angle such that the brace arm 118 is generally parallel to the first arm 114. In some embodiments, the brace arm can be rigidly coupled to the riser 120 so that the brace arm 118 remains at a fixed angle to the riser 120. In other embodiments, the brace arm 118 can be rotatably coupled to the riser 120 so that the brace arm 118 can pivot at its attachment point with the riser 120. In some of the pivoting embodiments, the brace arm's 118 pivoting movement is preferably restricted so that the brace arm 118 does not rotate past an approximately 90° angle to the riser 120. For example, as illustrated in the embodiment of FIGS. 23A-D, the brace arm 118 can have a stop member 190 that abuts against the second end region 148 of the riser 120 to prevent the brace arm 118 from rotating past an approximately 90° angle. In some embodiments, the brace arm 118 can be connected to the riser 120 by one or more pins, which can be removable. Preferably, the brace arm 118 is made of a rigid material, such as metals, composites and plastics. In some embodiments, the brace arm 118 can be made of a soft material that can deform under pressure. These embodiments advantageously enable the brace arm 118 to conform to the contours of the patient's body for added support.

The brace arm 118 can be a generally flat surface as illustrated in FIGS. 23A-D. In the illustrated embodiment, the brace arm 118 is rectangular in shape. However, in other embodiments, the brace arm 118 can be other shapes, such as circular, oval, square or polygonal. In some embodiments, the brace arm 118 can be configured to have a flat surface that faces the first arm 114, so that the brace arm 118 can be situated against a patient's body. In other embodiments, as described above, the brace arm 118 can be pre-shaped or can conform to the shape of the patient's body for added support.

In some embodiments the brace arm 118 can have openings or cutouts 119 on the surface. The cutouts 119 can provide weight reduction while maintaining sufficient rigidity and strength for compression support. In some embodiments, the brace arm 118 can have support ribs 192 that provide additional rigidity and support to the brace arm 118.

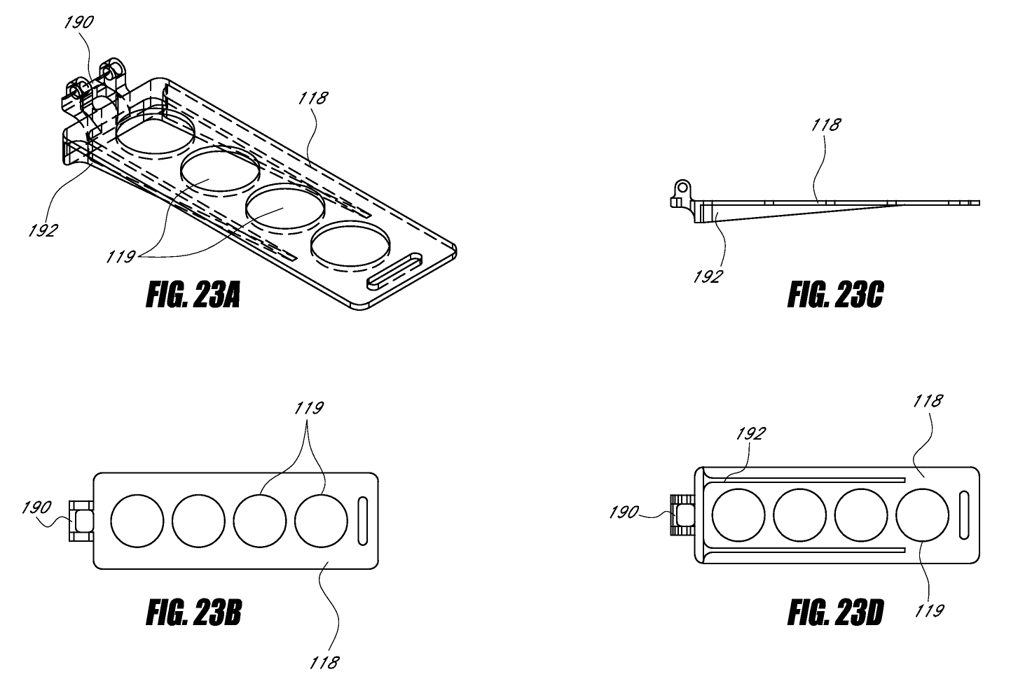

In some embodiments, a brace bushing 168, as illustrated in FIGS. 24A-B, can be inserted at the second end region 148 of the riser 120 and threaded by the second pin 162. The brace bushing 168 advantageously provides cushioning when the brace arm 118 is pivoted to the in use configuration. Preferably, the brace bushing 168 is made of an elastomeric material, such as rubber. However, other types of cushioning material can be used, such as for example, Styrofoam or soft plastics. In some embodiments, the brace arm 118 can be coupled to the riser 120 by other means, such as fasteners, hooks and grooves, or positioning apertures and projections, as described above.

Preferably, the components of the compression or tourniquet device 110 are made of biocompatible metals or hard plastics. For example, the riser and first arm can be made of stainless steel or aluminum and the pressure pad 150 can be made of Delrin. In other embodiments, the components can be made of any material having sufficient rigidity, such as fiberglass or composites. Although the components have been described as preferably being hollow or having cutouts for weight reduction, in some embodiments, the components can be at least partially or entirely solid. For example, the riser sleeve 136 and the first arm 114 can be solid metal or plastic rods.

In another embodiment of the first arm 114, instead of a flat portion 174 extending along the length of the first arm 114, a plurality of notches 194 can be disposed on the first arm 114, as illustrated in the embodiment of FIG. 25. The sleeve screw 129 can be tightened on the notches 194, allowing the first arm 114 to be secured at discrete positions relative to the sleeve adapter 126.

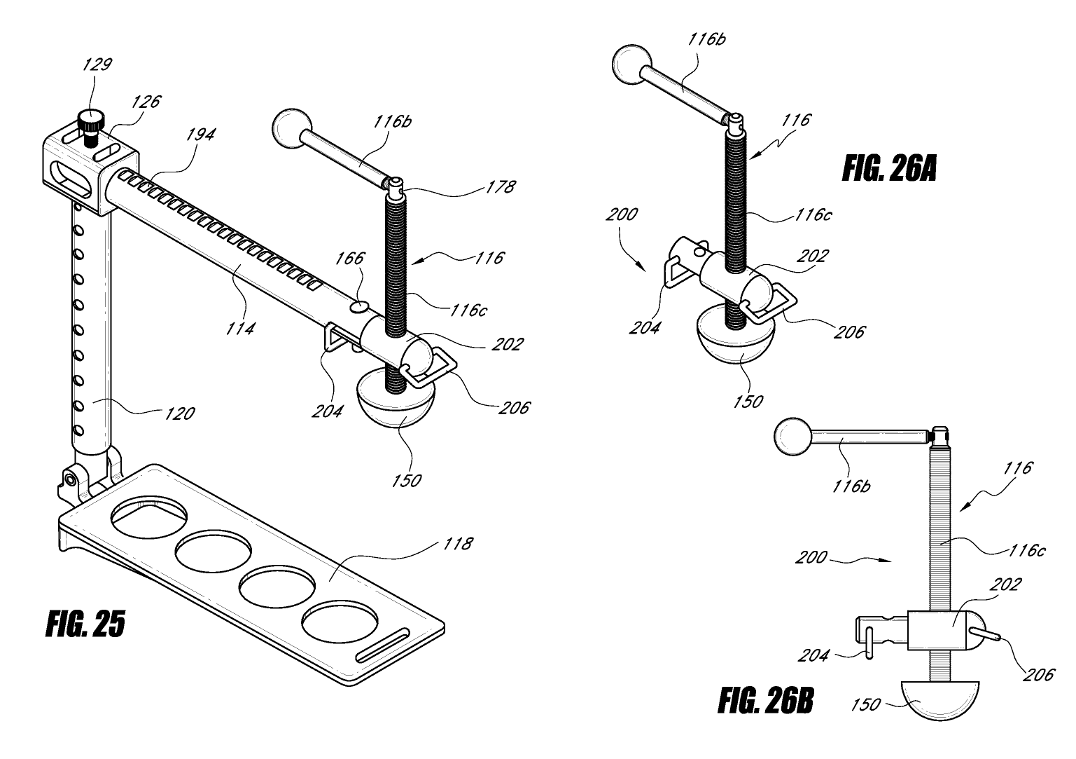

In some embodiments, illustrated in FIGS. 25 and 26A-B, the first arm 114 can comprise a spindle assembly 200 that can be removed and attached as a unitary piece. The spindle assembly 200 can comprise a first arm extension 202 having a square shaped first ring 204 that can be coupled to the second end region 115b of the first arm 114 and secured with a third pin 166, similar to the coupling of the ring 164. The spindle assembly 200 can comprise a spindle passage aperture 121 that can couple with a spindle 116. At an end region of the spindle 116 can be a handle mounting hole 178 where a handle 116b can attach. In the embodiment illustrated in FIGS. 25 and 26A-B, the handle 116b is connected to the handle mounting hole 178 with a threaded connection. At the head of the spindle 116c, is a pressure pad 150. In the illustrated embodiment, a square shaped second ring 206 is attached to the end region of the spindle assembly 200. The spindle assembly 200 can in some embodiments allow quick exchange of the pressure pad 150, so that various types of pressure pads can be readily used in different circumstances. The size of the injury or the placement of the injury on the patient's body can determine the size of the pressure pad 150 best suited for a situation.

### Method

To assemble the compression or tourniquet device 110, the spindle 116 can be inserted into the first arm 114 through the threaded spindle passage apertures 121. Once the head 116c of the shaft 116a emerges from the spindle passage aperture 120b, a pressure pad 150 is mounted onto the head of the shaft in a way that leaves the pressure pad freely rotatable on the shaft. By rotating the shaft 116a, the pressure pad 150 can be advanced or retracted relative to the brace arm 118.

The first arm 114 can be positioned in the sleeve member 128 and the sleeve screw 129 can be tightened to secure the position of the first arm 114 in the sleeve member 128. The brace arm 118 can be rotated to be at a generally 90° angle to the riser 120. The length of the riser 120 can be adjusted by depressing the projection(s) 143 and sliding the riser sleeve 136 until the projection(s) 143 aligns with a desired positioning apertures 142. Finally, in some embodiments, the riser 120 can be mounted to the sleeve adapter 126 by securing the first end region 144 of the riser 120 onto the mounting stud 130.

In use, some embodiments of the compression or tourniquet device 10, 110 can be used by a first responder or other medical personnel as a bleeding control device, such as a truncal tourniquet or compression device, to apply focused or direct compression or pressure to a body wound or opening, such as an uncontrollable pelvic, axillary and carotid hemorrhage, that may be otherwise untreatable with standard means currently available in the field today. The compression or tourniquet device 10, 110 can be placed over the point of injury where pressure can be evenly applied by the pressure pad 50, 150 until bleeding stops, by moving (e.g., screwing down) the pressure pad nearby or onto the wound. For example, for a front pelvic wound, the patient may be placed on his or her back, face up. The brace arm 18, 118 can be slipped under the patient's body beneath the wound, e.g., with the riser 20, 120 against the patient's side. The brace arm 18, 118 can be stationary with respect to the wound. The extender 38, 138 is mounted in the riser sleeve 36, 136 at a position that will place first arm 14, 114 at a suitable height over the patient's body. The first arm 14, 114 is secured in the sleeve adapter to position the pressure pad 50, 150 laterally relative to the riser 20, 120 so that the pressure pad is over the wound. The riser 20, 120 can provide adjustment of the vertical distance of the first arm 14, 114 and the pressure pad 50, 150 from the brace arm 18, 118, and that the first arm 14, 114 and the sleeve member 26, 126 provide adjustment of the lateral position of the first arm and pressure pad 50, 150 relative to the brace arm 18, 118. The position of the pressure pad 50, 150 is then adjusted (e.g., by moving it relative to the first arm 14, 114 and/or by adjusting the length of the riser 20, 120) to exert pressure against the bleeding wound to assist in staunching the blood-flow.

In some embodiments, the compression or tourniquet device 10, 110 can be mounted on a platform, such as for example a table, gurney, bed, stretcher, or carrying board. The compression or tourniquet device 10, 110 can be rigidly mounted to the platform. In some embodiments, the compression or tourniquet device can be removably mounted to the platform. In some embodiments, the compression or tourniquet device 10, 110 can be adjustable on the platform so that the device can be positioned over the injury on the patient. In some embodiments, the compression or tourniquet device 10, 110 may not require a brace arm 18, 118 for providing a lower support since the platform can provide the support. In some embodiments, the platform can have a pocket that accepts a portion, such as for example the brace arm 18, 118 for securing the compression or tourniquet device 10, 110 to the platform.

Initially, the spindle 16, 116 is generally in a retracted position, e.g., with the pressure pad 50, 150 near the first arm 14, 114. The user can then rotate or otherwise move the spindle 16, 116 by the handle 16b, 116b to advance the spindle through the first arm 14, 114 so that the pressure pad 50, 150 advances away from the first arm 14, 114, toward the brace arm 18, 118 and toward the wound. In some embodiments, a dressing is placed between the shaft 16a, 116a and the wound, and the spindle 16, 116 is advanced until the pressure pad 50, 150 makes contact with the dressing, or if there is no dressing, directly with the wound with sufficient pressure to stop or at least retard bleeding from the wound. The pressure pad 50, 150 may in some embodiments comprise a wound dressing, e.g., the pressure pad may comprise a metal plate having a gauze pad or other dressing fitted thereon or attached thereto.

A variety of interchangeable pressure pads 50, 150 of various sizes can be attached to the head 16c, 116c of the spindle 16, 116 so that the compression or tourniquet device 10, 110 can be equipped with a pressure pad 50, 150 of suitable size for the patient's wound. A large pressure pad 50, 150 is useful for applying disperse, even pressure onto a pressure bandage on a large wound, whereas a small ergonomic pressure pad may be designed for carotid bleeding and areas requiring more acute and deep pressure.

Once the patient's bleeding is under control, the first responder is free to attend to the immediate needs of other injured people.

The terms “first,” “second,” and the like, herein do not denote any order, quantity, or importance, but rather are used to distinguish one element from another. The terms “vertical” and “lateral” are used in a relative sense with respect to part of the claimed device and need not conform to an external frame of reference such as level ground. The terms “a” and “an” herein do not denote a limitation of quantity, but rather denote the presence of at least one of the referenced item.

Although particular embodiments have been described, numerous variations and alterations to the disclosed embodiments will be apparent form the foregoing disclosure. For examples, any of the features of the described embodiments can be combined in variations different from or in addition to those specifically described and/or illustrated.

The following is claimed:

1. A bleeding control device comprising:
a first frame member having a first end region and a second end region;
an adapter coupled to the first end region of the first frame member;
a second frame member being moveable with respect to the adapter, the second frame member being substantially perpendicular to the first frame member;
a support member coupled to the second end region of the first frame member, the support member being substantially parallel to the second frame member;
a shaft member attached to the second frame member, the shaft member being generally vertically adjustable on the second frame member and the shaft member being substantially perpendicular to the second frame member;
a pad disposed between the second frame member and the support member, the pad being connected to an end region of the shaft member;
wherein the distance from the pad to the support member is configured to be adjusted by manipulating the shaft member.

2. The device of claim 1, wherein the first frame member is adjustable in length.

3. The device of claim 1, wherein the device is configured to collapse for transport.

4. The device of claim 3, wherein the support member is pivotally connected to the second end region of the first frame member.

5. The device of claim 1, further comprising a fastener for coupling the second frame member to the adapter.

6. The device of claim 1, wherein the shaft member comprises a ball attached to the end region of the shaft member.

7. The device of claim 6, wherein the pad is rotatably and pivotally coupled to the ball.

8. The device of claim 1, wherein manipulating the shaft member involves screwing and unscrewing the shaft member on the second frame member.

9. The device of claim 1, wherein the pad comprises a wound dressing.

10. The device of claim 1, wherein the pad is interchangeable.

11. The device of claim 1, wherein the pad is semi-spherical in shape.

12. The device of claim 1, wherein the pad is generally rectangular in shape.

13. A bleeding control device comprising:
a frame comprising a first arm and a support arm; and
a pad coupled to the first arm;
wherein the first arm is generally laterally and generally vertically adjustable on the frame relative to the support arm.

14. The device of claim 13, further comprising a riser connected to the first arm at a first end region and connected to the support arm at a second end region, the second end region being opposite from the first end region on the riser, wherein the riser is configured to be adjustable in length so that the first arm is vertically adjustable relative to the support arm.

15. The device of claim 13, wherein the first arm is laterally adjustable relative to the riser.

16. The device of claim 13, further comprising a shaft member threaded through the first arm, wherein the pad is attached to an end of the shaft member.

17. The device of claim 13, wherein the device is configured to collapse for transport.

18. The device of claim 17, wherein the support arm is pivotally connected to the riser.

19. The device of claim 13, wherein the pad comprises a wound dressing.

20. The device of claim 13, wherein the pad is interchangeable.

21. The device of claim 13, wherein the pad is semi-spherical in shape.

22. The device of claim 13, wherein the pad is generally rectangular in shape.

23. A method of stopping bleeding from an injury of a patient, the method comprising the steps of:
positioning a bleeding control device near the injury so that a support arm of the device is under the patient's body beneath the injury;
adjusting a first arm of the device so that a pad coupled to the first arm is positioned over the injury;
manipulating a shaft member to move the pad closer to the support arm, providing pressure on the injury until the bleeding stops.
